(19) United States
(12) Patent Application Publication
(10) Pub. No.: US 2020/0345930 A1
(43) Pub. Date: Nov. 5, 2020

(54) PRESSURE INFUSION DEVICE FOR RETAINING INFUSION BAG AND ADJUSTABLE GRAVITY-INDEPENDENT OPERATION THEREOF

(71) Applicants: TAPMEDIC, LLC, Jacksonville, FL (US); BRIGHT ROOK MEDICA KFT., Debrecen (HU)

(72) Inventor: Richárd Puskás, Debrecen (HU)

(21) Appl. No.: 16/747,148

(22) Filed: Jan. 20, 2020

(30) Foreign Application Priority Data
May 3, 2019 (HU) P1900142

(51) Int. Cl. A61M 5/155; A61M 5/168; A61M 5/14

(52) U.S. Cl. CPC A61M 5/155; A61M 2205/3331; A61M 5/1413; A61M 5/16813

(57) ABSTRACT

Disclosed are various embodiments for a pressure infusion device, comprising a support unit formed of a flexible material and having a double-walled housing, the double-walled housing comprising a first wall and a second wall coupled to one another and defining a compartment therein, the support unit being configured to retain an infusion in the compartment, and a gas line having a first portion hermetically coupled to the first wall and a second portion hermetically coupled to the second wall such that pressure is applied to the infusion from both sides, the gas line being further coupled to an inflator.

### CROSS-REFERENCE TO RELATED APPLICATIONS

This application claims the benefit of and priority to Hungarian Patent Application No. P1900142 filed May 3, 2019, the contents of which being incorporated by reference in their entirety herein. This application is related to U.S. Patent application Ser. No. 29/718,001 filed Dec. 20, 2019, the contents of which being incorporated by reference in their entirety herein.

### TECHNICAL FIELD

This invention belongs to the technical field of infusion bags and, more specifically, relates to a pressure unit and a support unit with a pressure relief device for fixing an infusion bag and for controlling an infusion system in a gravity-independent, adjustable manner which can be mounted on a human body or other near flat surface.

### BACKGROUND

Classical solutions for infusions typically use the force of gravitational attraction to deliver therapeutic solution to vasculature. When using such systems, a container or plastic bag containing an infusion, such as blood, is assembled with an infusion tube assembly. For instance, an infusion tube assembly is connected by a cannula inserted into a vein, artery, or bone. The tube assembly contains a drop chamber, which, when partially filled with the solution, makes it possible to watch the flow of liquid falling in droplets as a result of gravity and its approximate velocity. With these systems, the infusion bag must be positioned above the human body during operation; otherwise, due to the laws of transport vessels, the infusion tube may draw blood toward the infusion bag, which may endanger the patient or expel air to the partially air-containing drip chamber and veins.

As such, when using traditional infusion systems, close monitoring is required at least because, when the infusion bag is empty, the vessel in which the cannula is placed and the space formed by the tube assembly forms an air pocket. In addition to the flow of blood, in the event of a venous vacuum is formed, an air embolism can be created by drawing air into the veins from the inside of an already empty tube assembly. The tube assembly and infusion container require careful attention when moving patients due to the aforementioned issues. In on-field and on-field conditions, this extra focus will take away power and time from the care worker and carry additional risks.

Existing pressure infusion systems, by manual injection of air into an elastic gas hose cuff or by inflating with a gas cartridge, operate by applying pressure to accelerate the flow of fluids into the bloodstream. These systems do not provide any benefit to the user or patient other than accelerating the flow. Pipe fittings provided in traditional systems include a risk of air entering the bloodstream due to use of a drip chamber. During use, the infusion bag should be kept above the patient's body line in order to prevent possible accidents. Continuous monitoring is required as classic infusion sets are completely emptied after the therapeutic solution in the infusion bag is depleted.

Due to applied pressure, any air remaining in the infusion bag is pushed into the bloodstream. In the absence of a non-return valve, the empty tubing can draw blood back from the veins and maintain an open connection between the space of the fixture and the patient's circulation. Whenever the patient is to be moved, a suitable stand or person is required to maintain the dose above the patient's body line and ensure that the pressurized element of the system does not tilt, overturn, and/or deliver air into the patient's veins from the space of a drip chamber directly adjacent to it. In most cases, these solutions include an inflatable hose made of rubber or silicone, which is tightly seated next to the infusion bag and enclosed in a common, inflexible wallet. This will provide the necessary overpressure around the infusion bag.

In the related art, U.S. Pat. No. 4,657,160 describes a control unit that includes a manually operated valve that allows pressurized gas to inflate the pressure cuff to force the solution out of the bag and to allow the pressure cuff to rapidly vacuum.

Chinese Patent No. 2,139,450 describes a soft-packaged liquid pressurized infusion device consisting of a pressurized canister and a pressurized gas bag, wherein a plug door is formed on the pressure canister. The suspension loop is located at the top of the overpressure box. The bottom of the pressurized box is provided with a hole for the passage of an infusion catheter. A hole is formed along the side wall of the pressurized container to pass a gas bag tube. The upper and lower parts of the gas bag are provided with an adjusting valve and a one-way air inlet valve. The advantages of the soft-pressurized liquid-pressurized infusion set include simple structure, convenient operation, freely adjustable infusion rate, ease of work for medical staff and one-on-one operation.

U.S. Pat. No. 6,699,234 discloses light, thin, and flexible drug infusion devices. The devices are characterized in that each of them has a plurality of reservoir bodies, a pump, a pump controller, one or more batteries/accumulators placed on a flexible insert so that the devices can be glued to a user's skin like a large and thick wound patch. In the first embodiment, each container body contains a medicament pouch that contains the medicament. The rest of the tank bodies contain air. By blowing air into the container bodies, the drug is compressed from the drug pouch into the user's body. In the second embodiment, each tank body contains medicine and an air bag. Blowing air into the air bags delivers the drug from the container bodies to the user's body. The third embodiment is similar to the second, where the drug is pressurized from the container bodies into the user's body. The fourth embodiment is similar to the first, where the drug is released from the drug bag into the body by pressure.

International Patent Application No. PCT/HU2016/000034 discusses a pre-filled pressurized infusion set. The pre-filled pressurized infusion set consists of an infusion bag, a conduit, a flow controller, preferably at least one drug delivery port, and a non-return valve. The line has a flow indicator, the flow indicator has an inlet connector and an outlet connector, a turbine housing, a turbine and preferably a cover plate, the turbine shaft being loosely fitted by the bearing bores of the turbine housing and cover plate. The conduit has at least one curved portion, preferably a spiral, the infusion bag is in a pressure pocket, the infusion bag is surrounded by at least four sides by the pressure pocket, and there is a pneumatic cuff between the pressure pocket and the infusion bag; which is connected to a changeover valve and a pump, the pressure valve has a memory foam layer. The memory foam layer has a self-adhesive coating with a peelable protective film. The pressure pocket is at least partially transparent, preferably of mesh material, and the pneumatic cuff is at least partially transparent. The line is pre-filled with the infusion solution, the line is connected to the infusion bag by a continuous connection, and the stopcock has a removable closure.

The disadvantage of this solution includes the memory foam mounting with the mounting plate placed above it not being flexible enough and not being able to follow the curves of different human body surfaces with sufficient flexibility. Changes in the skin surface that wrinkle during patient movement cannot be tracked and the risk of peeling increases. Furthermore, after the surface fixation used in this solution and after the infusion has been completed, the entire system should be replaced in case of possible additional solution as the system is completely indestructible. The amount of gas produced by the cartridge is chemically constant. Correction is not possible with decreasing or increasing external pressure. Gas cannot be replaced.

Generally speaking, the disadvantages of known solutions is that infusion bags are pressed only from a single side so that the infusion bag can easily be wound and folded, and thus the therapeutic solution contained therein cannot flow out completely. Further, the design of known solutions and prior systems are somewhat complicated and costly to manufacture.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a top plan view of a pressure infusion device having a support unit according to various embodiments of the present disclosure.

FIG. 2 is a bottom plan view of the pressure infusion device of FIG. 1 according to various embodiments of the present disclosure.

FIG. 3 is a plan view of a mounting surface according to various embodiments of the present disclosure.

FIG. 4 is a front view of the support unit of the pressure infusion device of FIG. 1 according to various embodiments of the present disclosure.

FIG. 5 is a front view of the pressure infusion device according to various embodiments of the present disclosure.

FIG. 6 is a longitudinal cross-sectional view of a unit containing two infusion bags according to various embodiments of the present disclosure.

FIG. 7 is a top perspective view of the pressure infusion device having the support unit according to various embodiments of the present disclosure.

FIG. 8 is a bottom perspective view of the pressure infusion device of FIG. 7 according to various embodiments of the present disclosure.

FIG. 9 is a top view of the pressure infusion device of FIG. 7 according to various embodiments of the present disclosure.

FIG. 10 is a bottom view of the pressure infusion device of FIG. 7 according to various embodiments of the present disclosure.

FIG. 11 is a front view of the pressure infusion device of FIG. 7 according to various embodiments of the present disclosure.

FIG. 12 is a rear view of the pressure infusion device of FIG. 7 according to various embodiments of the present disclosure.

FIGS. 13 and 14 are side views of the pressure infusion device of FIG. 7 according to various embodiments of the present disclosure.

FIGS. 15-20 are photographs illustrating an example use of the pressure infusion device according to various embodiments of the present disclosure.

### DETAILED DESCRIPTION

The patent application relates to a pressure infusion device unit having a support unit for fixing an infusion bag and controlling an infusion system in a gravity-independent, adjustable manner, where the pressure infusion device can be mounted on a human body or other near flat surface. An infusion bag can be positioned in the support unit to receive equal pressure from both sides during infusion, and the flow rate of infusion fluid in the infusion bag can be reliably controlled, and optionally accelerated or decelerated.

The present invention overcomes shortcomings in conventional systems by providing an infusion bag holder unit that can be placed within or below the line of the human body and simply and securely mounted on a nearly flat surface, even on the patient's body. An infusion bag described herein can be symmetrically pressed from both sides such that the flow rate of the infusion fluid in the infusion bag can be reliably controlled, optionally accelerated or decelerated, and the infusion solution set to flow as the patient moves or moves almost unchanged. The flow rate of infusion fluid flowing out of the infusion bag in the support unit is ensured and is independent of gravity, maintaining the benefits of using overpressure when using the system and eliminating all of the dangers listed above and utilizing other practical options all at once. As such, the infusion bag holder described herein can be widely used.

The use of a gravity independent pressurized support unit with its special infusion set, which signals flow through a special turbine instead of a drip chamber, achieves an unattended design that reduces the burden on suppliers. Recognizing the potential of the device to accommodate up to two infusion bags at the same time and to provide the patient with a doubled amount of fluid, or simultaneously deliver two different solutions to the human bloodstream via an implanted, inserted, or drilled cannula, needle, or via a catheter without automatically applying hydrostatic pressure due to gravitational pull which automatically stops and closes the vein due to a stop valve due to failure due to malfunctioning, vacuum formation in the patient's veins for safe and minimal monitoring applications.

Accordingly, a pressure infusion device is described as having a pressure gauge and control unit for securing an infusion bag and for controlling the infusion system in a gravity-independent, adjustable manner, where an infusion bag containing an infusion fluid, and a tubular end portion attached thereto, is a standard part of an infusion system and ends in a cannula. The tube assembly is provided with a flow control, at least one drug delivery port, a non-return valve and a flow indicator, and a turbine. In the present invention, the infusion bag can be housed in a support unit consisting of two overlapping cross-sections, symmetrically overlapping housing sides and a pressure gauge and pressure relief assembly, such that the gas lines exiting in the housings are connected to a pressure gauge, and a pressure relief assembly and a valve are connected.

The two walls of the support unit are connected to each other on at least two sides, preferably on their longitudinal sides, and the open portion enclosed by the two pouches is a compartment into which an infusion bag can be placed. The walls are of a flexible material, having two walls and forming a closed airspace within themselves.

A gas line can be hermetically connected to both housings, the other ends of the gas lines being connected to a single pole of a three-pole connector, and the third line of the connector being connected to a gas line that is connected to the pressure gauge and pressure limiter. The gas line can be connected to the lower part of the pressure gauge and pressure relief unit, and also connected to a pump with a valve.

The pressure measuring and pressure limiting unit is connected to the gas line and its cylindrical housing includes a piston. The lower part of the piston in the housing has a silicone sealing ring which seals the space of the housing under the piston, the lower part of the housing having a hole. The inside wall of the house has a longitudinal groove in the longitudinal direction which starts at the top edge of the house, but does not reach the bottom of the house. The purpose of milling is to release air through the silicone sealing ring by intentional leakage, thereby controlling the maximum air pressure in the system. In the housing, the piston is surrounded by a spring and secured by a closure cap to the housing. The lower part of the spring is fixed above the silicone sealing ring and the upper pole is fixed on the inner surface of the cap. The milling ends at a height above the bottom of the housing that reaches the height of the piston silicone ring against the spring when the air pressure inside the gas pipe and casing rises to 400 mmHg (53328,955 Pa).

In the lower part of the housing, there is a material-connected connection piece with two housing outlets, one outlet connected to the gas line to the connection unit and the other outlet to the gas line to the rubber pump. With this solution, the pump, the gas lines, the pressure gauge and pressure relief unit, and the two housings form a solid gas space.

The infusion bag placed in the support unit is secured by attaching the closure tabs to a closure by attaching the closure tabs secured to the lower and upper portions of the lower enclosure to a hook-and-loop fastener on the outer surface of the enclosure top case. The compartment is thus partially closed, keeping the infusion bag stable.

The size of the unit is characterized by the fact that an infusion bag of a given volume can be easily inserted into the compartment. Suitably, the double-walled, closed-wall enclosures of the flexible material have the shape of an infusion bag, preferably rectangular. On the lower surface of the support unit, on the outer surface of the lower case, there is an adhesive element provided with a protective layer. The adhesive member fits well to the adhesive surface of the mounting surface. The adhesive element and the attachment surface are fastened to each other with a fastener. The attachment surface is fixed to the human body or to any near-flat surface by means of an adhesive.

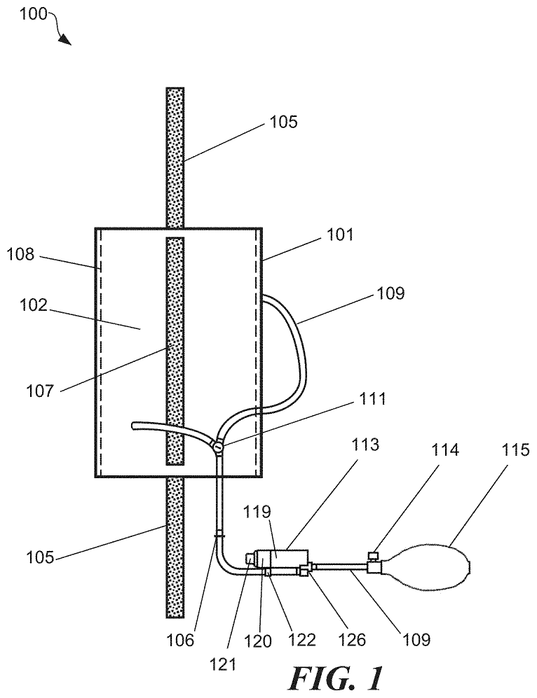

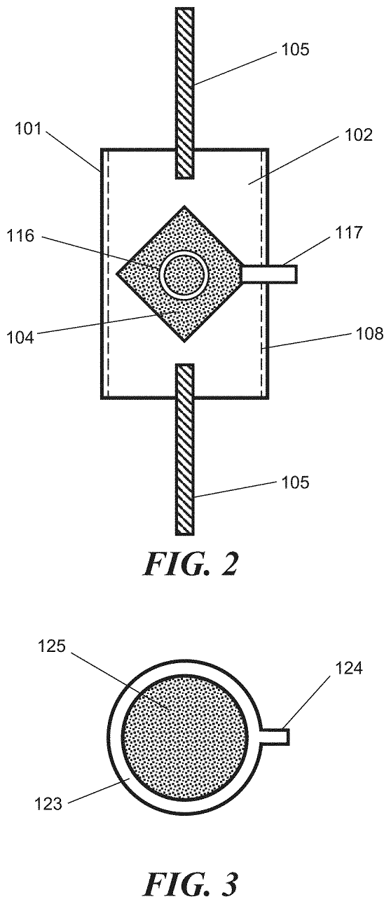

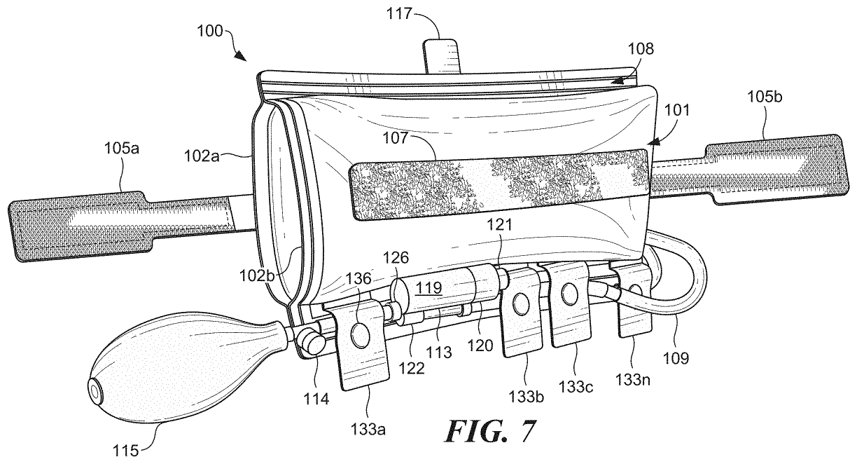

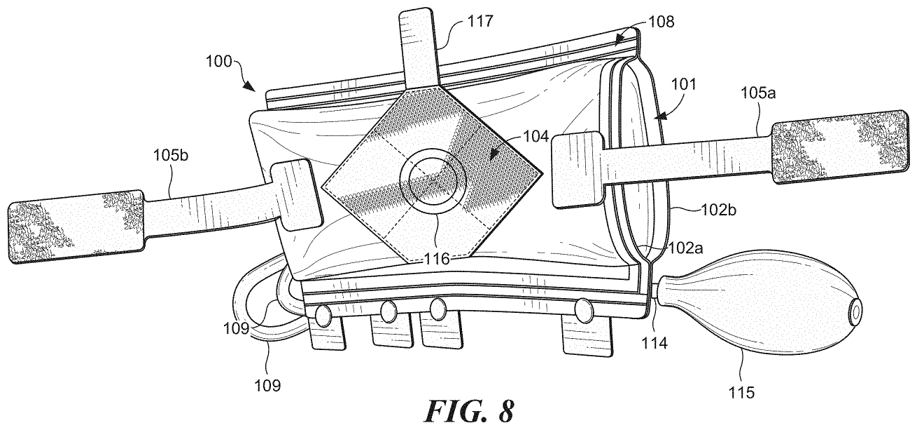

Turning now to the figures, FIG. 1 is a top plan view of a pressure infusion device 100 and FIG. 7 is a top perspective view of the pressure infusion device 100. Referring to these figures collectively, the pressure infusion device 100 includes a support unit 101. In some embodiments, the support unit 101 comprises an air-filled double-walled housing configured to retain an infusion bag 110 or other infusion device, generally referred to herein as an infusion, where the infusion bag 110 is shown in FIGS. 2 and 8. An infusion bag 110 can include a traditional infusion bag comprising a liquid, such as blood, solution, or other substance as can be appreciated. The double-walled housing of the support unit 101 can include a first wall 102a and a second wall 102b (collectively “walls 102”). As the walls 102 of the support unit 101 symmetrically overlap, only the first wall 102a (e.g., a top wall) is shown in FIG. 1. However, both the first wall 102a and the second wall 102b are shown in FIG. 7.

Notably, the second wall 102b (e.g., the top wall) comprises a closure 107 (or a “closing mechanism”), such as a hook-and-loop fastener, which can be secured in a longitudinal direction in a central part thereof. Locking tabs 105a, 150b (collectively “locking tabs 105”) project laterally from the support unit 101 such that the locking tabs 105 extend beyond outermost edges of the walls 102. The locking tabs 105 can include an end secured to the first wall 102a (e.g., the bottom wall) and can be releasably attached to the closure 107. The locking tabs 105 can include a flexible and bendable body, as can be appreciated. Similar, the walls 102 of the support unit 101 can be formed of a flexible material, such as plastic or other flexible polymer, and can be double-walled to form a closed air space. For instance, the walls 102 can be joined to one another along their longitudinal side at a connecting strip 108, which can include sewing, welding, or other suitable connection mechanisms.

The walls 102 can include apertures 130 on their outer surfaces, respectively, through or to which a gas line 109 (also referred to as “tubing”) is hermetically connected. In some embodiments, ends of the gas lines 109 can be connected to a connector unit 111 (e.g., a three-pole connector unit 111), shown in FIG. 1, which may be a three-way or a Y-connector in some embodiments. The other end of the gas line 109 can be connected to a third pole of the connector unit 111, and can be provided with a pressure gauge and pressure control unit 113 connected to the gas line 109 in some embodiments. A retaining ring 122 can secure the pressure gauge and pressure control unit 113 to the gas line 109. The pressure gauge and pressure control unit 113 can be connected to a pump 115 provided with a valve 114 via a gas line 109. The pump 115 can include a rubber pump in some embodiments.

In various embodiments, the pressure gauge and pressure control unit 113 can include a cylindrical housing 119, and can further include a piston 121. In the housing 119, the piston 121 can be surrounded by a spring (not shown) and can be secured by a closure cap 120. A connection piece 126 in a lower part of the housing 119, which is formed in a continuous manner with the housing 119, can include two outlets, where one outlet is connected to the gas line 109 to the connector unit 111, and the other outlet to the gas line 109 to the pump 115. As shown in FIG. 1, the connecting element 106 serves to connect and, if necessary, quickly disconnect the gas lines 109 to eliminate overpressure in the entire system.

FIG. 2 and FIG. 8 are bottom views of the support unit 101. Referring to these figures collectively, on the longitudinal sides of the first wall 102a of the support unit 101, a connecting strip 108 is shown as well as the locking tabs 105 and their attachment to the shorter outer side of the first wall 102a. A fastener 104 (e.g., a hook-and-loop or Velcro® fastener, or an adhesive element) can be secured to the outer surface of the first wall 102a. In some embodiments, the fastener 104 (e.g., an adhesive element) can be formed of two overlapping layers, the lower layer being the same as the material of the walls 102, the overlapping layer (as shown) having a hook-and-loop material (e.g., a Velcro® material). The two layers can be secured to one another by sewing or other suitable coupling, and the fastener 104 thus formed can be secured to the outer surface of the first wall 102a by a fastening strip 116, such as a circular fastening strip, which can be sewn or welded. A targeting separator projection 117 connected to the edge of the fastener 104 can be made of a continuous material from the lower (non-Velcro) layer. The separator projection 117 allows the protective layer that covers the fastener 104 to be easily removed during use.

FIG. 3, which is a plan view of a mounting surface, shows the attachment surface 123 having a tear-off projection 124 and a central portion having an adhesive surface 125. When fitted to the attachment surface 123, the targeting separator projection 117 of the fastener 104 is closely aligned with a tear-off projection 124 to direct the engagement, thereby ensuring that the hook-and-loop closure of the fastener 104 is centered on the adhesive surface 125 of the attachment surface 123.

The side of the attachment surface 123 opposite to the adhesive surface 125 is provided with an adhesive which is covered with a protective layer or protective film during packaging and transport. The adhesive can secure the attachment surface 123 to as many surfaces as possible.

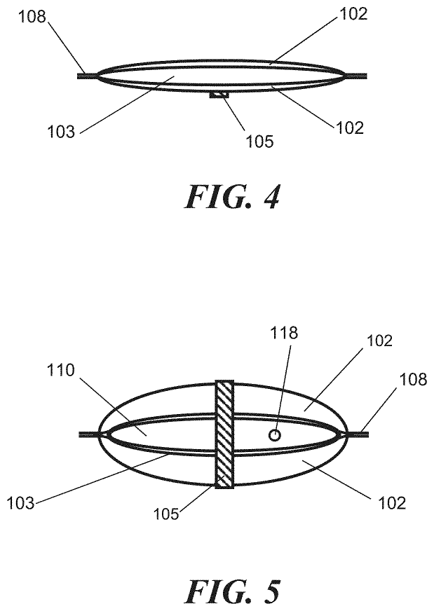

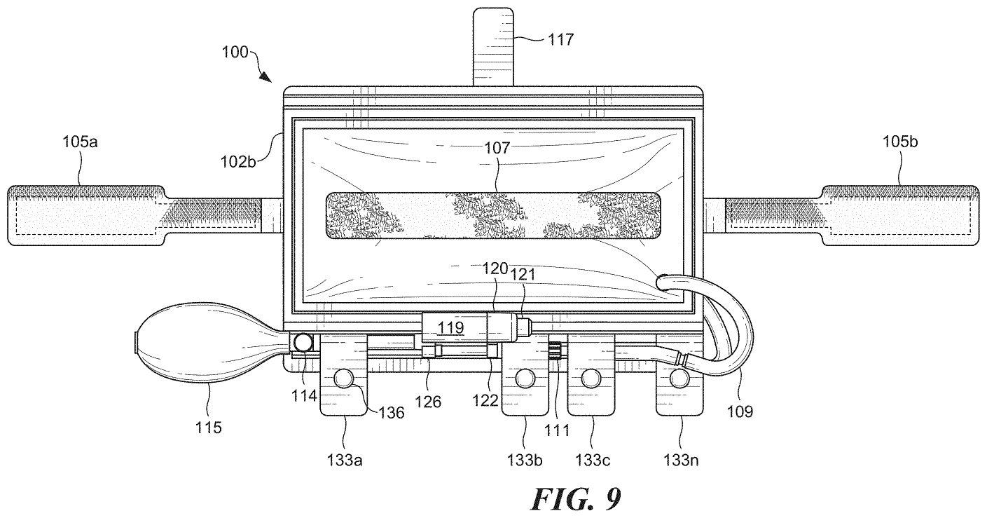

FIG. 4 and FIG. 9 are front views of the support unit 101. Referring to FIG. 4 first, the wall 102 and the compartment 103 are shown as being defined by its inner surfaces, and the locking tabs 105 in the open position in normal condition (without pressure), not filled with air. FIG. 9 shows the support unit 101 of the pressure infusion device 100 being inflated with air, which can be performed by squeezing the pump 115, directing air through the gas lines 109 into the interior of the support unit 101, as can be appreciated.

FIG. 5 is a front view of the pressure infusion device which shows air-filled, pressurized walls 102 and an infusion bag 110 in a compartment 103, the drain port 118 of which being covered by a closure when not in use and an infusion tube assembly being connected to a drain port 118 when in use. FIG. 5 shows that the locking tabs 105 at least partially closes the compartment 103, holding an infusion bag 110 positioned therein securely.

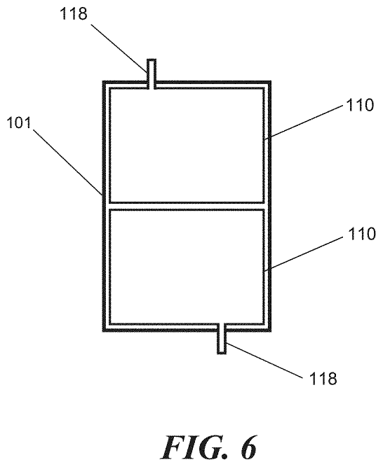

In FIG. 6, two infusion bags 110 are shown placed in a support unit 101. The infusion bags 110 are disposed opposite to each other in the compartment 103 and a plurality of tubing and fittings can be connected to ports 118 of the infusion bags 110, as can be appreciated. According to one or more embodiments, two ports 118 can be connected on both sides of the support unit 101, which can be connected to a gas line 109, where the gas line 109 can be one connected to the connector unit 111, such as a Y-shaped connector unit. For example, a first end of a gas line 109 can be connected to the third pole of the connector unit 111 and a second end thereof can be connected to the pressure measuring and pressure control unit 113. The pressure gauge and pressure control unit 113 can be connected to a pump 115 provided with a valve 114 by another gas line 109. An infusion bag 110 can be placed in the support unit 101 which can completely fill the compartment 103, such as a 500 ml infusion bag. The infusion bag 110 can be fixed in the compartment 103 without moving, by attaching the locking tabs 105 attached to a lower one of the walls 102 to the closure 107 on an upper one of the walls 102.

According to a further embodiment, the support unit 101 has the same configuration as in the previous example, but the compartment 103 is fitted with two infusion bags 110 having the same volume as the infusion bag 110 in the previous example. The two infusion bags 110 can be disposed opposite to each other, with a discharge on one of the ports 118 facing outwards. According to a further embodiment, the infusion bags 110 are also placed in the support unit 101, but are located on each other facing in the opposite direction to the ports 118.

Referring again to FIG. 7, in some embodiments, the pressure infusion device 100 can include a plurality of attachment tabs 133a . . . 133n. The attachment tabs 133 can extend downward, in some embodiments, permitting the gas line 109 and other various components of the pressure infusion device 100 to be tightly secured to the support unit 101, creating a compact-type pressure infusion device 100 that can be beneficial in combat and other emergency situations. The attachment tabs 133 can include a button 136 or other attachment mechanism that secures the attachment tabs 133 to a bottom portion of the support unit 101. While FIG. 7 includes four attachment tabs 133, the pressure infusion device 100 can include one, three, or another suitable number of attachment tabs 133.

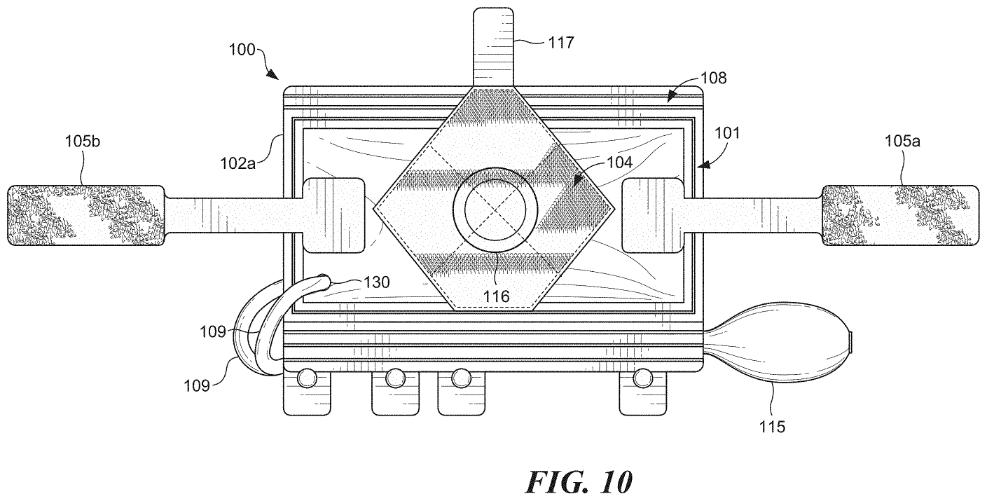

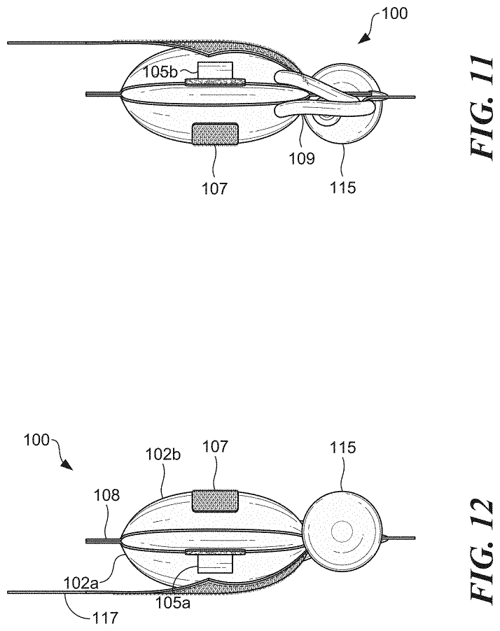

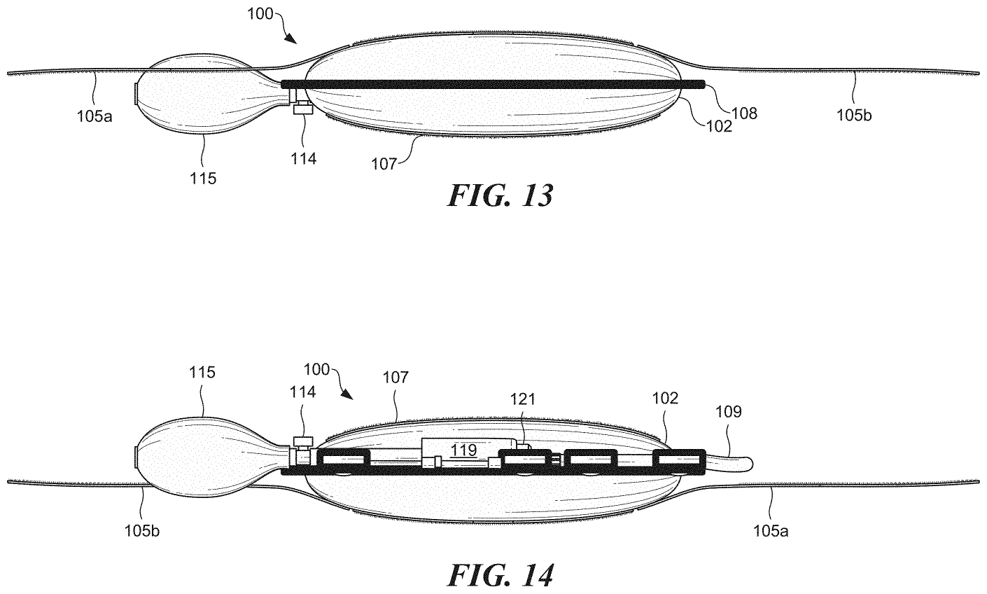

FIG. 10 shown a bottom view of the pressure infusion device 100 according to various embodiments of the present disclosure. FIG. 11 shows a front view of the pressure infusion device 100 according to various embodiments of the present disclosure. FIG. 12 is a rear view of the pressure infusion device 100 according to various embodiments of the present disclosure. Also, FIGS. 13 and 14 are side views of the pressure infusion device 100 according to various embodiments of the present disclosure.

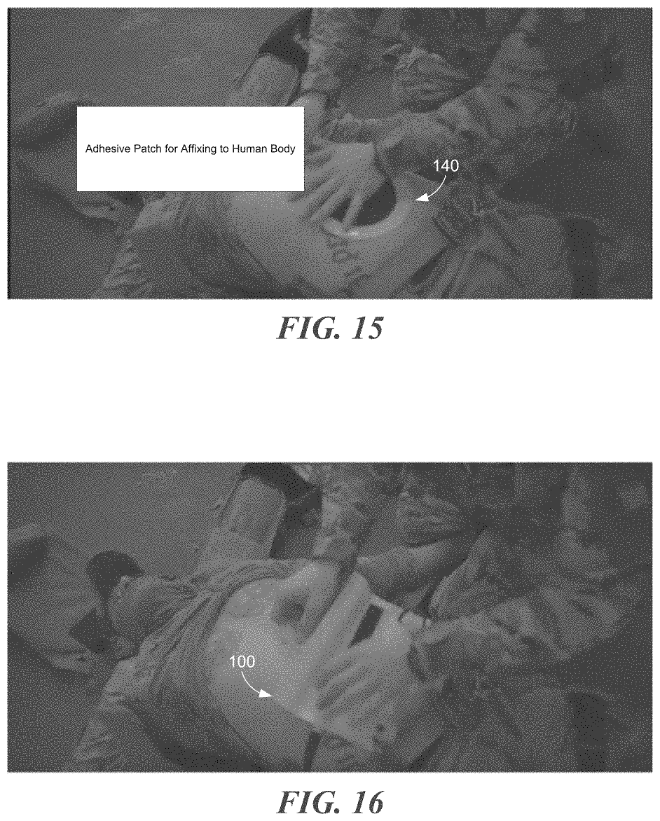

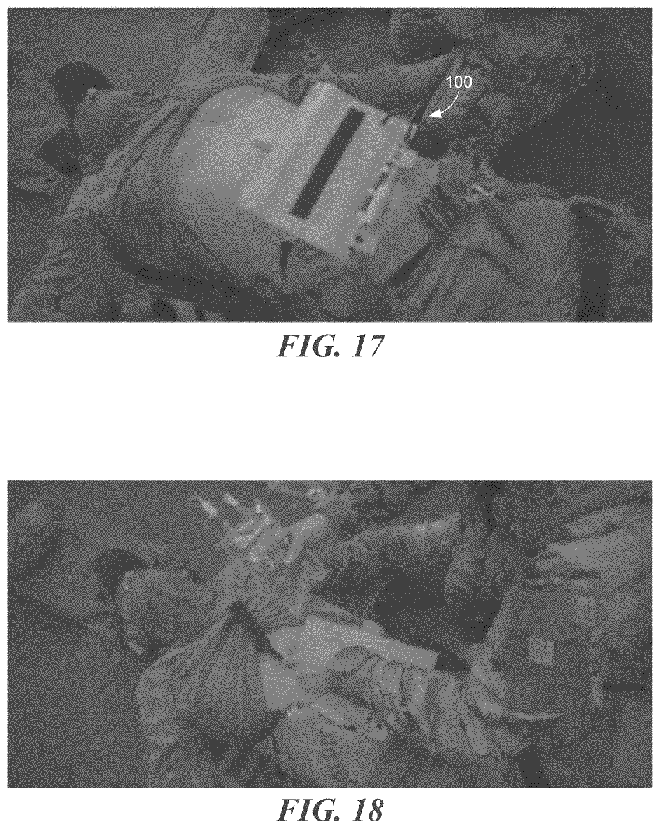

Referring now to FIGS. 15-20, an example application of the pressure infusion device 100 to a human is described. The present invention allows an infusion bag fixed in the support unit 101 to be equally pressurized on both sides when infused. When using the pressure infusion device 100 described herein, a first step can include affixing an adhesive patch 140 to a human body. An inner surface of the adhesive patch 140 (e.g., the surface contacting skin of the human shown in FIG. 15) can include an adhesive that adheres to human skin. An outer surface of the adhesive patch 140 can include a hook-and-loop fastener or other connection for coupling to the pressure infusion device 100. Next, as shown in FIG. 16 and FIG. 17, the pressure infusion device 100 can be secured to the adhesive patch 140, thereby securing the pressure infusion device 100 to the human body. The pressure infusion device 100 is shown as non-inflated in these figures.

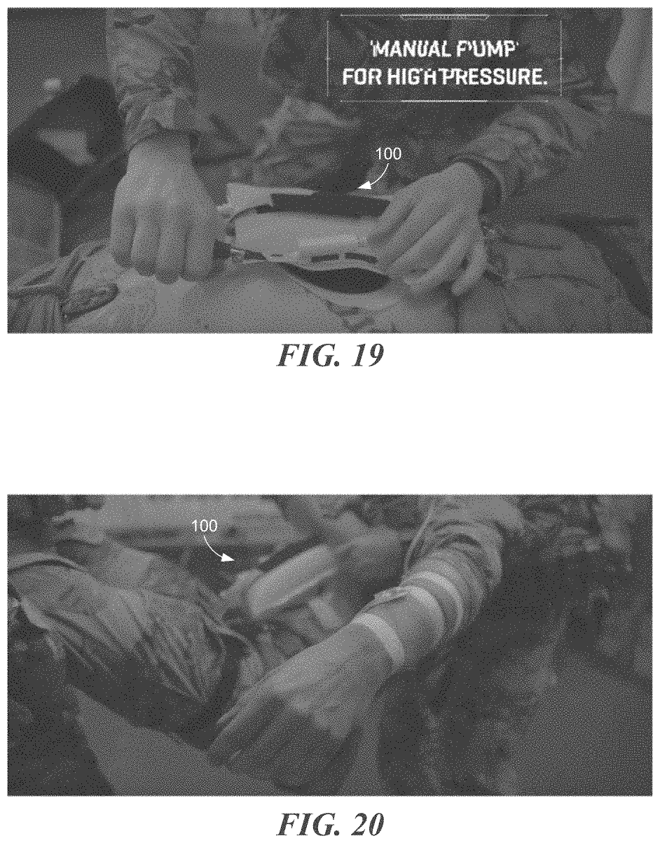

Next, as shown in FIG. 18, an infusion, such as an intravenous (IV) bag, is inserted into the pressure infusion device 100 or, more specifically, into the compartment defined by the walls 102 of the support unit 101. In FIG. 19, the pump 115 is pumped by hand, inflating the double-walled housing of the support unit 101, equally pressurizing both sides of the IV bag or other infusion. In FIG. 20, as the pressure infusion device 100 operates in a gravity-independent manner, the patient can be moved, for example, to a hospital. The IV bag fixed in the support unit 101 receives equal pressure from both sides during infusion, and the flow rate of infusion fluid in the infusion bag can be reliably controlled, optionally accelerated, or decelerated.

The pressure infusion device 100 can be placed within or below the line of the human body and simply and securely mounted on a nearly flat surface, such as the stomach of the patient's body. The infusion bag described herein can be symmetrically pressed from both sides such that the flow rate of the infusion fluid in the infusion bag can be reliably controlled, optionally accelerated, or decelerated, and the infusion solution set to flow as the patient moves or moves almost unchanged. The flow rate of fluid flowing out of the infusion bag in the support unit 101 can be controlled and is independent of gravity, maintaining the benefits of using pressure when using the system, eliminating various dangers.

Another example operation is described as follows. For example, a first step can include connecting a tube assembly to an infusion bag after removing a closure cap. The second step can include venting the resulting system and filling the tube assembly with therapeutic solution. The third step can include removing a protective layer from an attachment surface and securing it with an adhesive-coated surface to a patient's body or on another near flat surface. The fourth step includes inserting the infusion bag into the compartment of the support unit 101. The fifth step can include securing the infusion bag to the adhesive surface of the attachment surface by securing the infusion bag in the compartment by fastening the closing tabs with a hook-and-loop fastener, such as Velcro®. A cannula is connected to the appropriate body portion of the patient, thereby providing a venous input to the patient's body. After closing the valve, the support unit 101 can be inflated using the pump 115.

By pumping air, both walls 102 of the support unit 101 are equally saturated with air and the pressure in the support unit 101 is controlled by a pressure gauge and control unit. As the pressure in the pressure infusion device 100 increases, the pressure entering the pressure gauge and limiter unit through a bore exerts an increased force on the bottom of the piston 121 and the seal, which initially prevents air from escaping and increasing pressure further.

Increasing pressure can push the piston 121 out of the housing 119 against the spring. The spring can be selected so that it approximately allows, for instance, as a pressure of 400 mmHg (53328,955 Pa), the lower pole of the plunger to raise so that a seal there reaches the lowest point of the milling cut. In this case, gas escapes to the outside of the gasket at the milling point and prevents further pressure increase. The milling length and spring force are set so that the piston is 400 mmHg (53328,955 Pa), so high that it reaches the milling mill and hence causes leakage to protect the system from overpressure.

The pressure infusion device 100 of the present invention can deliver therapeutic solutions to the human bloodstream through a cannula, needle, or catheter implanted, inserted, or drilled without the use of hydrostatic pressure due to gravitational attraction and with the appropriate (check valve and flow turbine). With the use of a fitted infusion tubing, the flow of the infusion fluid stops automatically and the check valve closes the vein, due to a stop valve that supplies a patient's vein at the end of dosing or when a malfunction occurs due to a malfunction or vacuum. The pressure infusion device 100 can be quickly attached to the human body by a suitable fastening procedure, allowing the patient to stand up and move without interrupting the administration of the solution.

All these benefits make the system particularly suitable for use in combat and on-site emergency patient care in low health care settings.

The features, structures, or characteristics described above may be combined in one or more embodiments in any suitable manner, and the features discussed in the various embodiments are interchangeable, if possible. In the following description, numerous specific details are provided in order to fully understand the embodiments of the present disclosure. However, a person skilled in the art will appreciate that the technical solution of the present disclosure may be practiced without one or more of the specific details, or other methods, components, materials, and the like may be employed. In other instances, well-known structures, materials, or operations are not shown or described in detail to avoid obscuring aspects of the present disclosure.

Although the relative terms such as “on,” “below,” “upper,” and “lower” are used in the specification to describe the relative relationship of one component to another component, these terms are used in this specification for convenience only, for example, as a direction in an example shown in the drawings. It should be understood that if the device is turned upside down, the “upper” component described above will become a “lower” component. When a structure is “on” another structure, it is possible that the structure is integrally formed on another structure, or that the structure is “directly” disposed on another structure, or that the structure is “indirectly” disposed on the other structure through other structures.

In this specification, the terms such as “a”, “an”, “the”, and “said” are used to indicate the presence of one or more elements and components. The terms “comprise,” “include,” “have,” “contain,” and their variants are used to be open ended, and are meant to include additional elements, components, etc., in addition to the listed elements, components, etc. unless otherwise specified in the appended claims. The terms “first”, “second”, etc. are used only as labels, rather than a limitation for a number of the objects.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A pressure infusion device, comprising:
a cannula, at least one flow regulator, and a valve assembly;
a support unit formed of a flexible material and having an air-filled double-walled housing to which a gas line is hermetically connected, the support unit being configured to retain an infusion bag in a compartment of the air-filled double-walled housing;
a three-pole coupling unit, ends of the gas line being connected to a first pole and a second pole of the three-pole coupling unit;
a pressure gauge and pressure control unit mounted on the gas line and connected to a third pole of the three-pole coupling unit, and further connected to a rubber pump with a valve positioned therebetween;
the support unit having two walls that are symmetrically overlapping and having at least two sides of which are fixed to one another such that the infusion bag can be placed in a compartment defined between the two walls;
at least one edge of a first one of the two walls comprising a locking tab projecting laterally beyond the support unit, a surface of the locking tab being provided with a fastening element;
an outer surface of a second one of the two walls being provided with a hook-and-loop fastener, wherein the hook-and-loop fastener is configured to secure the infusion bag in the compartment through attachment of the locking tab to the hook-and-loop fastener; and
the pressure infusion device being configured to apply pressure to the infusion bag equally from both sides.

2. The pressure infusion device according to claim 1, wherein the two walls are connected to one another at a connecting strip.

3. The pressure infusion device according to claim 1, where the fastening element is an adhesive element consisting of two layers, an outer layer of which having a hook-and-loop closure and a separating projection projecting from the adhesive element.

4. The pressure infusion device according to claim 3, wherein the adhesive element is attached to the outer surface of the second one of the two walls by way of a circular fastening strip.

5. The pressure infusion device according to claim 3, wherein the adhesive element is connected to a fixing surface having a projection on a human body.

6. The pressure infusion device according to claim 1, wherein the compartment is sized and positioned to retain at least two infusion bags at the same time.

7. A pressure infusion device, comprising:
a support unit formed of a flexible material and having a double-walled housing, the double-walled housing comprising a first wall and a second wall coupled to one another and defining a compartment therein, the support unit being configured to retain an infusion in the compartment; and
a gas line having a first portion hermetically coupled to the first wall and a second portion hermetically coupled to the second wall such that pressure is applied to the infusion from both sides, the gas line being further coupled to an inflator.

8. The pressure infusion device according to claim 7, further comprising:
a cannula, at least one flow regulator, and a valve assembly;
a three-pole coupling unit comprising a first pole, a second pole, and a third pole, wherein ends of the gas line are connected to the first pole and the second pole;
a pressure gauge and pressure relief unit mounted on the gas line and connected to the third pole of the three-pole coupling unit and further connected to a rubber pump with a valve positioned therebetween;
at least one edge of a first one of the two walls comprising a locking tab projecting laterally beyond the support unit, a surface of the locking tab being provided with a fastening element; and
an outer surface of a second one of the two walls being provided with a hook-and-loop fastener, wherein the pressure and pressure relief device is configured to secure an infusion bag in the compartment through attachment of the locking tab to the hook-and-loop fastener.

9. A method, comprising:
providing a pressure infusion device, comprising:
a support unit formed of a flexible material and having a double-walled housing, the double-walled housing comprising a first wall and a second wall coupled to one another and defining a compartment therein, the support unit being configured to retain an infusion in the compartment; and
a gas line having a first portion hermetically coupled to the first wall and a second portion hermetically coupled to the second wall such that pressure is applied to the infusion from both sides, the gas line being further coupled to an inflator.

10. The method of claim 9, further comprising:
a cannula, at least one flow regulator, and a valve assembly;
a three-pole coupling unit comprising a first pole, a second pole, and a third pole, wherein ends of the gas line are connected to the first pole and the second pole;
a pressure gauge and pressure relief unit mounted on the gas line and connected to the third pole of the three-pole coupling unit and further connected to a rubber pump with a valve positioned therebetween;
at least one edge of a first one of the two walls comprising a locking tab projecting laterally beyond the support unit, a surface of the locking tab being provided with a fastening element; and
an outer surface of a second one of the two walls being provided with a hook-and-loop fastener, wherein the pressure and pressure relief device is configured to secure an infusion bag in the compartment through attachment of the locking tab to the hook-and-loop fastener.
